(19) United States
(12) Patent Application Publication
(10) Pub. No.: US 2021/0087224 A1
(43) Pub. Date: Mar. 25, 2021

(54) COMPOSITIONS AND METHODS FOR GENERATING MODIFIED CRYO POOR PLASMA

(71) Applicant: PLASMA TECHNOLOGIES, LLC, Charleston, SC (US)

(72) Inventors: Eugene ZURLO, Charleston, SC (US); Dennis CURTIN, Plattsburgh, NY (US); Peter RADTKE, Charleston, SC (US); Kurt L. BRILLHART, Mission Viejo, CA (US)

(21) Appl. No.: 16/738,656

(22) Filed: Jan. 9, 2020

Related U.S. Application Data
(60) Provisional application No. 62/903,613, filed on Sep. 20, 2019.

(51) Int. Cl. C07K 1/16; A01N 1/02

(52) U.S. Cl. CPC C07K 1/16; A01N 1/0284; A01N 1/021

(57) ABSTRACT

A method for producing a modified cryo-poor precipitate that can be utilized in chromatography without intervening precipitation steps is provided. While thawing frozen plasma at low temperature a precipitating compound (e.g. a salt of an organic acid) is added in small amounts. The resulting modified cryo-poor plasma has a reduced tendency to foul chromatography media, permitting direct application to such media without the need for additional precipitation steps. The resulting modified cryoprecipitate has a higher content of cold-insoluble proteins (such as clotting factors), and can be resolubilized and processed further.

This application claims the benefit of U.S. Provisional Patent Application No. 62/903,613 filed on Sep. 20, 2019. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is blood products, in particular serum or plasma

### BACKGROUND

The background description includes information that may be useful in understanding the present invention. It is not an admission that any of the information provided herein is prior art or relevant to the presently claimed invention, or that any publication specifically or implicitly referenced is prior art.

The non-cellular portion of human blood has long been used as a source of human proteins for therapeutic use (such as immunoglobulins, albumin, clotting factors, alpha 1 antitrypsin, etc.). Currently the most common source is blood plasma obtained from commercial donation centers. Such proteins need to be isolated at high purity, while minimizing denaturation. In order to be successful commercial endeavors need to recover these proteins in high yield and purity by scalable processes.

Unfortunately, these practical (e.g. purity) and commercial (e.g. yield) requirements are frequently at odds with one another. In order to achieve high purity it is often necessary to use chromatographic techniques, such as ion exchange, hydrophobic interaction, affinity, size exclusion, or mixed-mode chromatography. Serum and plasma, however, have very high protein concentrations and also include a significant amount of denatured material generated during storage. This generally results in fouling of the chromatography media due to nonspecific binding and/or entrapment if serum or plasma is applied directly. Such fouling decreases the rate of flow through the column and ultimately decreases the capacity of the chromatography media, and may not be removable by extensive washing.

One approach to resolving this issue is to either dilute the source blood product or subject it to one or more precipitation steps in order to reduce the protein concentration. Dilution, however, necessarily increases the volume of material that needs to be processed, and necessitates the use of large volumes of chromatography media. While precipitation (such as Cohn fractionation) can reduce the protein burden, some precipitants (notably ethanol), along with changes in pH, temperature, and/or extended processing time, can denature sensitive proteins. In addition, poor selectivity during precipitation can result in loss of a portion of the desired protein to the uncollected fraction.

Thus, there remains a need for a rapid, simple, and non-denaturing approach to treating serum and/or plasma in therapeutic protein isolation.

### SUMMARY OF THE INVENTION

Methods and compositions of the inventive concept provide a modified cryoprecipitation procedure that generates a modified cryo-poor plasma preparation suitable for direct application to chromatography without intervening precipitation steps.

One embodiment of the inventive concept is a method for making a modified cryo-poor plasma, by thawing frozen plasma at a temperature of from about 1° C. to about 6° C. in the presence of a precipitant to generate a modified cryoprecipitate and a modified cryo-poor plasma. The precipitant is supplied in a concentration that does not result in formation of a visible precipitate when the concentration is provided to non-frozen plasma. The resulting modified cryoprecipitate can then be separated from the modified cryo-poor plasma. Suitable precipitants include, but are not limited to, organic acids, salts of organic acids (such as sodium citrate), inorganic salts, and hydrophilic polymers. The resulting modified cryoprecipitate can have an increased content of cold-insoluble proteins relative to a conventional cryoprecipitate generated by thawing the frozen plasma at a temperature of from about 1° C. to about 6° C. in the absence of the precipitant. Similarly, the modified cryo-poor plasma has a decreased content of cold-insoluble proteins or denatured proteins relative to a conventional cryo-poor plasma generated by thawing the frozen plasma at a temperature of from about 1° C. to about 6° C. in the absence of the precipitant.

Another embodiment of the inventive concept is a method for isolating a protein from plasma, by thawing frozen plasma at a temperature of from about 1° C. to about 6° C. in the presence of a precipitant to generate a modified cryoprecipitate and a modified cryo-poor plasma, separating the modified cryoprecipitate from the modified cryo-poor plasma, applying the modified cryo-poor plasma to a chromatography media without an intervening precipitation, filter separation by molecular weight/size (e.g. ultrafiltration, diafiltration), and/or significant dilution step to produce an unbound and bound fractions, and recovering the protein of interest (e.g. albumin, and immunoglobulin, alpha-1 antitrypsin) from either the unbound fraction or the bound fraction. The precipitant can be supplied in a concentration that does not result in formation of a visible precipitate in non-frozen plasma. Suitable precipitants include organic acids, salts of organic acids (such as sodium citrate), inorganic salts, and hydrophilic polymers. The modified cryoprecipitate so produced can have an increased content of cold-insoluble proteins relative to a conventional cryoprecipitate generated by thawing the frozen plasma at a temperature of from about 1° C. to about 6° C. in the absence of the precipitant. Similarly, the modified cryo-poor plasma can have a decreased content of cold-insoluble proteins or denatured proteins relative to a conventional cryo-poor plasma generated by thawing the frozen plasma at a temperature of from about 1° C. to about 6° C. in the absence of the precipitant. Suitable chromatography media include but are not limited to size exclusion media, ion exchange media, hydrophobic interaction media, affinity media, and mixed-mode media. In some embodiments the modified cryoprecipitate is further processed by solublizing the cryoprecipitate and isolating a second protein (e.g. fibrinogen, fibronectin, a clotting factor) from the cryoprecipitate.

Another embodiment of the inventive concept is a process intermediate that includes a precipitant, a modified cryoprecipitate (which includes an increased content of cold-insoluble proteins relative to a conventional cryoprecipitate generated by thawing the frozen plasma at a temperature of from about 1° C. to about 6° C. in the absence of the precipitant), and a modified cryo-poor plasma (which includes a decreased content of cold-insoluble proteins or denatured proteins relative to a conventional cryo-poor plasma generated by thawing the frozen plasma at a temperature of from about 1° C. to about 6° C. in the absence of the precipitant). Suitable precipitants include, but are not limited to, organic acids, salts of organic acids (such as sodium citrate), inorganic salts, and hydrophilic polymers. Typical cold-insoluble proteins include fibrinogen, fibronectin, and clotting factors.

### BRIEF DESCRIPTION OF THE DRAWINGS

FIG. 1 schematically depicts an embodiment of a method of the inventive concept.

### DETAILED DESCRIPTION

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The source material for the vast majority of blood products is frozen plasma obtained from commercial collection centers. Slowly thawing this material at low temperature (typically from 1 to 6° C.) generates an intermediate blood product that contains precipitated proteins (i.e. cryoprecipitate or “cryo”) and a protein-rich supernatant (cryo-poor plasma). Cryoprecipitate includes some of the fibrinogen content of the source plasma, as well as clotting factors and fibrin. Cryo-poor plasma is rich in cold-soluble proteins and is frequently used as a source of pharmaceutical proteins.

Cryo-poor plasma has a protein content and/or denatured protein content that renders it unsuitable for direct application to conventional chromatographic separations without an intervening dilution or processing step. Surprisingly, the Inventors have found that the inclusion of a low concentration of a precipitant (i.e. a concentration that does not result in observable precipitation when applied to serum and/or plasma) in the thawing process can alter the protein distribution between cold-soluble and cold-insoluble fractions in the resulting preparation. The resulting modified cryo-poor plasma has been found to have a protein content that permits direct application to chromatography media (e.g. size exclusion media, ion exchange media, hydrophobic interaction media, affinity media, mixed-mode chromatography media, etc.) without intervening dilution, preparative filtration, and/or precipitation steps. In a preferred embodiment of the inventive concept the chromatography media is an affinity media. This advantageously both simplifies and reduces the time and materials required for plasma processing time. In addition, reduction in the number of processing steps can reduce the degree to which sensitive protein species are denatured, resulting in improved stability and specific activity.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

Without wishing to be bound by theory, the Inventors believe that fouling of chromatography media can be due, at least in part, to the presence of fibrinogen/fibrin and/or other non-target proteins in blood products (e.g. plasma) used in the isolation of therapeutic proteins. Inventors believe that when provided at low temperatures during a thawing process low concentrations of a precipitant can act to selectively reduce the solubility of cold-insoluble proteins such as fibrinogen, while retaining solubility of commercially valuable cold-soluble proteins.

In some embodiments of the inventive concept the precipitant is provided in amounts that do not generate observable precipitate when applied to serum or plasma. In other embodiments of the inventive concept the precipitant is provided in amounts that generate observable precipitate when applied to serum or plasma.

The amount of precipitant used can vary depending upon the nature of the precipitant. Suitable precipitants are preferably nondenaturing, and can include organic acids and salts of organic acids (e.g. sodium citrate), inorganic salts (e.g. ammonium sulfate, sodium sulfate, sodium chloride), and hydrophilic polymers (e.g. PEG, dextran, etc.). For example, if an organic salt such as sodium citrate is used it can be provided during thawing of frozen plasma at concentrations ranging from about 1%, 2%, 3%, 4%, 5%, 6%, 7%, 8%, 9%, 10%, 12%, 15%, 18%, or less than about 20% (w/v). Similarly, if an inorganic salt such as ammonium sulfate is used it can be provided during thawing of frozen plasma at concentrations ranging from about 1%, 2%, 3%, 4%, 5%, 6%, 7%, 8%, 9%, 10%, 12%, 15%, 18%, or less than about 20% (w/v). If a hydrophilic polymer such as PEG is used it can be provided during thawing of frozen plasma at concentrations ranging from about 0.1%, 0.2%, 0.5%, 0.7%, 1%, 1.5%, 2%, 2.5%, 3%, 4%, 5%, 6% 8%, or less than about 10% (w/v).

In some embodiments of the inventive concept a precipitant can be provided as a dry solid, preferably a powder or crystalline powder, that is added directly to the thawing plasma. In other embodiments a precipitant can be provided as a concentrated stock solution, which can be added in volumes sufficiently small as to not significantly impact the volume of the thawing plasma. For example, such a stock solution can contain the precipitant at a concentration that is from 5-fold to 100-fold higher than the desired final concentration. In some of such embodiments the precipitant can be added gradually while mixing. In such embodiments the concentration of the precipitant in the precipitating mixture gradually increases throughout the volume of the mixture until the final desired concentration is reached. In other embodiments the precipitant can be added as a single bolus. In such embodiments a region of the precipitating mixture near the point of addition can transiently have concentrations of the precipitant that are greater than the target concentration, however undesired precipitate resulting from such transient elevations will solvate as the concentration of precipitant equilibrates.

Addition of a precipitant at the low concentration noted above during thawing results in generation of a modified cryoprecipitate and a modified cryo-poor plasma. The modified cryoprecipitate has a different and distinct composition from that of a cryoprecipitate formed from the same source plasma without the addition of the precipitant. For example, the modified cryoprecipitate can have an increased amount of fibrin, fibrinogen, or denatured/partially denatured proteins relative to a corresponding conventional cryoprecipitate. As such modified cryoprecipitate can be a more desirable product for isolation of cold-insoluble proteins (such as fibrinogen, clotting factors, etc.) than conventional cryoprecipitate, for example providing higher yield per unit of frozen plasma.

Accordingly, a modified cryo-poor plasma can have a different and distinct composition from that of a conventional cryo-poor plasma produced by cryoprecipitation of the same source plasma in the absence of the precipitant. For example, the modified cryo-poor plasma can have a reduced content of fibrinogen, fibrin, and/or denatured or partially denatured proteins relative to conventional cryo-poor plasma without significantly impacting the content of cold-soluble proteins. This reduced protein content can permit application of such a modified cryo-poor plasma directly to a chromatography media without intervening precipitation and/or significant dilution steps. In some embodiments, depending upon the nature or the chromatography media minor dilution of modified cryo-poor plasma may be required. In such embodiments cryo-poor plasma can be diluted from about 1:1.1, 1:1.25, 1:1.5, 1:1.75, 1:2, 1:2.5, or up to about 1:3 prior to application to the chromatography media.

Chromatography media can have any suitable formulation and configuration. Suitable chromatography media can be formulated to provide separation by size exclusion ion exchange, hydrophobic interaction, affinity, and/or mixed mode interactions. Suitable media can be provided as porous granules or beads, non-porous granules or beads, filters, fibers, and/or porous membranes. Structural portions of chromatography media can be based on any suitable materials. Examples include but are not limited to polysaccharides (such as cross-lined dextran), synthetic polymers, and/or inorganic materials (such as hydroxyapatite). Chromatography media can be provided in any suitable geometry. Suitable geometries include open or sealed chromatography columns, radial chromatography columns, cartridges, membrane housings, etc. In some embodiments granular or bead-based chromatography media can be added directly to a modified cryo-poor plasma as a loose material, rather than being contained in a device or enclosure through which the modified cryo-poor plasma is passed.

Due to the lack of fouling use of an excessively large amount of chromatography media is not required. For example, when a granular or particulate media is used the volume ratio of modified cryo-poor plasma to chromatography media can range from about 1:1 to 1:20, depending upon the protein to be recovered and the capacity of the chromatography media for the protein.

It should be appreciated that lack of fouling can also reduce the need for extensive and/or harsh washing conditions (e.g. high or low pH, high or low salt concentrations, surfactants, enzymes, etc.) for regeneration of chromatography media following use. This can advantageously reduce the time required for regeneration, thereby improving turnaround time in what is frequently a bottleneck step in protein purification processes. Similarly, the ability to use relatively mild washing/regeneration conditions can increase the number of useful cycles for a chromatography media (particularly affinity chromatography media). This advantageously reduces the need to replace or replenish the media.

In some embodiments modifications to the cryo-poor plasma may be necessary prior to application to a chromatography media in order to meet the pH or ionic strength requirements for the media's separation mode. For example, pH may need to be adjusted by the addition of a small volume of acid or base prior to application to ion exchange media. Similarly, ionic strength may need to be reduced (e.g. by dialysis or the use of desalting media) prior to application to ion exchange media. Similarly, ionic strength may need to be increased (for example, by the addition of a salt) prior to application to hydrophobic interaction media.

A variety of pharmaceutically useful proteins can be obtained from methods of the inventive concept at high yield and specific activity. Such proteins include fibrinogen, factor VII, factor VIII, factor IX, factor XIII, von Willebrand factor, fibronectin, immunoglobulins, alpha-1 antitrypsin, protein C, protein S, C1-esterase inhibitor, antithrombin III, thrombin, and/or albumin.

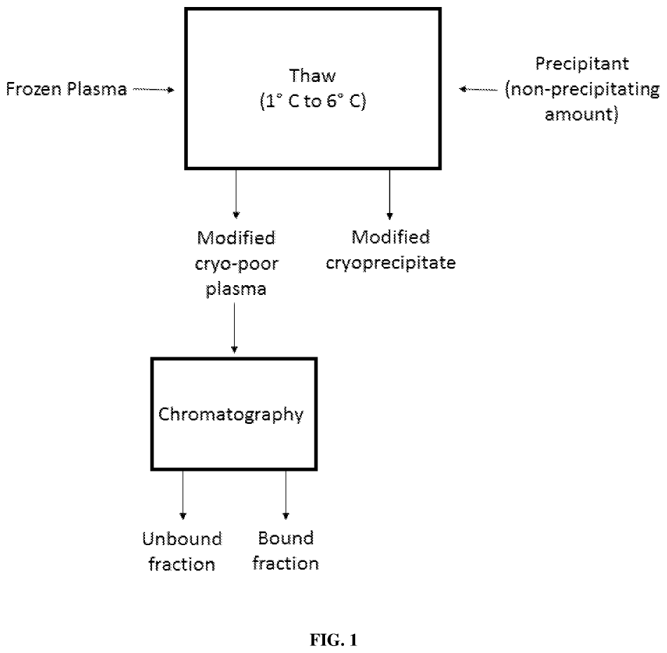

In an example of a method of the inventive concept (as shown in FIG. 1), a unit of frozen plasma is obtained and allowed to thaw at a temperature of from 1° C. to 6° C. During thawing sodium citrate is added (either by addition of dry sodium citrate or by a small volume of a 50% (w/v) sodium citrate stock solution) to give a final concentration of 8% or less. Following the completion of thawing and the formation of modified cryoprecipitate, the modified cryo-poor plasma is separated from solid materials (i.e. the modified cryoprecipitate). This can be accomplished by filtration, centrifugation, decanting, or any suitable technique. The modified cryoprecipitate can be recovered and solubilized for further processing and recovery of pharmaceutical proteins that are relatively cold-insoluble (e.g. fibrinogen, factor VIII, factor XIII, von Willibrand factor, and/or fibronectin). The recovered cryo-poor plasma is applied to an affinity chromatography media without intervening precipitation, preparative filtration (e.g. molecular weight or size specific filtration), and/or dilution steps to recover pharmaceutically valuable proteins that are relatively cold soluble. For example, the modified cryo-poor plasma can be applied to a protein-A or protein-G functionalized affinity chromatography media without intervening precipitation or dilution steps, then the media washed and eluted with a low pH buffer to recover immunoglobulin G. Similarly, a procion blue functionalized affinity chromatography media can be used to recover albumin. In some embodiments an affinity chromatography media functionalized with an antibody specific for a desired pharmaceutically valuable protein can be used to recover the desired protein. In preferred embodiments such an antibody is directed to alpha-1 antitrypsin.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. A method of providing a modified cryo-poor plasma, comprising:
obtaining a first volume of frozen plasma;
thawing the first volume of frozen plasma at a temperature of from about 1° C. to about 6° C. to generate a thawing plasma;
adding an amount of a precipitant to the thawing plasma to generate a modified cryoprecipitate and a modified cryo-poor plasma, wherein the amount is selected to not generate a precipitate when the amount of the precipitant is added to a second volume of thawed plasma, wherein the second volume is equivalent to the first volume; and
separating the modified cryoprecipitate from the modified cryo-poor plasma.

2. The method of claim 1, wherein the precipitant is selected from the group consisting of an organic acid, a salt of an organic acid, an inorganic salt, and a hydrophilic polymer.

3. The method of claim 1, wherein the precipitant is sodium citrate.

4. The method of claim 1, wherein the modified cryoprecipitate has an increased content of cold-insoluble proteins relative to a conventional cryoprecipitate generated by thawing the frozen plasma at a temperature of from about 1° C. to about 6° C. in the absence of the precipitant.

5. The method of claim 1, wherein the modified cryo-poor plasma has a decreased content of cold-insoluble proteins or denatured proteins relative to a conventional cryo-poor plasma generated by thawing the frozen plasma at a temperature of from about 1° C. to about 6° C. in the absence of the precipitant.

6. (canceled)

7. A method of isolating a protein from plasma, comprising:
obtaining a volume of frozen plasma;
thawing the frozen plasma at a temperature of from about 1° C. to about 6° C. in the presence of a precipitant to generate a modified cryoprecipitate and a modified cryo-poor plasma;
separating the modified cryoprecipitate from the modified cryo-poor plasma;
applying the modified cryo-poor plasma to a chromatography media without an intervening precipitation or significant dilution step to produce an unbound fraction and a bound fraction; and
recovering a first protein from either the unbound fraction or the bound fraction.

8. The method of claim 7, wherein the precipitant is selected from the group consisting of an organic acid, a salt of an organic acid, an inorganic salt, and a hydrophilic polymer.

9. The method of claim 7, wherein the precipitant is sodium citrate.

10. The method of claim 7, wherein the modified cryoprecipitate has an increased content of cold-insoluble proteins relative to a conventional cryoprecipitate generated by thawing the frozen plasma at a temperature of from about 1° C. to about 6° C. in the absence of the precipitant.

11. The method of claim 7, wherein the modified cryo-poor plasma has a decreased content of cold-insoluble proteins or denatured proteins relative to a conventional cryo-poor plasma generated by thawing the frozen plasma at a temperature of from about 1° C. to about 6° C. in the absence of the precipitant.

12. The method of claim 7, comprising selecting an amount of the precipitant such that the precipitant is supplied in a concentration that does not result in formation of a visible precipitate when the concentration is provided to a volume of non-frozen plasma equivalent to the volume of frozen plasma.

13. The method of claim 7, wherein the chromatography media is an affinity media.

14. The method of claim 7, comprising the steps of:
collecting the modified cryoprecipitate;
solubilizing the cryoprecipitate; and
isolating a second protein from the cryoprecipitate.

15. The method of claim 7, wherein the first protein is selected from the group consisting of albumin, and immunoglobulin, and alpha-1 antitrypsin.

16. The method of claim 14, wherein the second protein is selected from the group consisting of fibrinogen, fibronectin, and a clotting factor.

17. A process intermediate, comprising:
a precipitant,
a modified cryoprecipitate comprising an increased content of cold-insoluble proteins relative to a conventional cryoprecipitate generated by thawing the frozen plasma at a temperature of from about 1° C. to about 6° C. in the absence of the precipitant; and
a modified cryo-poor plasma comprising a decreased content of cold-insoluble proteins or denatured proteins relative to a conventional cryo-poor plasma generated by thawing the frozen plasma at a temperature of from about 1° C. to about 6° C. in the absence of the precipitant.

18. The process intermediate of claim 17, wherein the precipitant is selected from the group consisting of an organic acid, a salt of an organic acid, an inorganic salt, and a hydrophilic polymer.

19. The process intermediate of claim 17, wherein the precipitant is sodium citrate.

20. The process intermediate of claim 17, wherein cold-insoluble proteins comprise at least one of the group consisting of fibrinogen, fibronectin, and a clotting factor.

21. The method of claim 1, wherein the precipitant is added in dry form.
